(19) United States
(12) Patent Application Publication
(10) Pub. No.: US 2022/0336066 A1
(43) Pub. Date: Oct. 20, 2022

(54) COUNSELING AUTOMATION PROVIDING EVIDENCE OF CONCORDANCE

(71) Applicant: IKYN Inc., Wilmington, DE (US)

(72) Inventors: Hal M. Spitz, Oakland, CA (US); Gary Fleischer, Raleigh, NC (US); Eric Kiebler, Half Moon Bay, CA (US)

(21) Appl. No.: 17/723,041

(22) Filed: Apr. 18, 2022

Related U.S. Application Data
(60) Provisional application No. 63/201,204, filed on Apr. 18, 2021.

(51) Int. Cl. G16H 10/60; G06F 11/34; G06F 21/31

(52) U.S. Cl. CPC G16H 10/60; G06F 11/3438; G06F 21/31; G06Q 50/18

(57) ABSTRACT

An apparatus for counseling automation providing evidence of concordance includes a processor and non-transitory computer readable storage media storing code. The code is executable by the processor to perform operations that include recording telemetry data of a consumer playing one or more media files. Each media file includes information being disclosed to the consumer. Each media file is associated with a unique media identifier. Each media file and an associated unique media identifier are stored on an immutable data storage system. The telemetry data includes information indicative of the consumer being exposed to the media files while the media files are being played. The operations include creating an electronic fingerprint of the telemetry data and the unique media identifiers of the one or more media files and storing the electronic fingerprint, the telemetry data and the unique media identifiers of the one or more media files in a ledger.

### CROSS-REFERENCES TO RELATED APPLICATIONS

This application claims the benefit of U.S. Provisional Patent Application No. 63/201,204 entitled “Counseling Automation Providing Irrefutable Indication Of Concordance” and filed on Apr. 18, 2021 for Gary Fleischer, et al., which is incorporated herein by reference.

### FIELD

This invention relates to proof of counseling and more particularly relates to an automated counseling application providing evidence of concordance.

### BACKGROUND

Often a consumer is asked to view disclosure information for various reasons. For example, a patient may be asked to view media files with disclosure information prior to a medical procedure, prior to taking medication, etc. In other environments, a consumer may be asked to review regulations, rules, responsibilities, etc. In other instances, a consumer may be asked to review warranty information before taking possession of a product. Often lawsuits and other disputes result from the consumer stating that the information in the media files was never disclosed to the consumer.

### SUMMARY

An apparatus for counseling automation providing evidence of concordance is disclosed. A method and computer program product also perform the functions of the apparatus. The apparatus includes a processor and non-transitory computer readable storage media storing code. The code is executable by the processor to perform operations N that include recording telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system. The telemetry data includes information indicative of the consumer being exposed to the one or more media files while the one or more media files are being played. The operations include creating an electronic fingerprint of the telemetry data and the unique media identifiers of the one or more media files and storing the electronic fingerprint, the telemetry data, and the unique media identifiers of the one or more media files in a ledger.

A method for counseling automation providing evidence of concordance includes recording telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system. The telemetry data includes information indicative of the consumer being exposed to the one or more media files while the one or more media files are being played. The method includes creating an electronic fingerprint of the telemetry data and the unique media identifiers of the one or more media files and storing the electronic fingerprint, the telemetry data, and the unique media identifiers of the one or more media files in a ledger.

A program product for counseling automation providing evidence of concordance includes a non-transitory computer readable storage medium storing code. The code is configured to be executable by a processor to perform operations that include recording telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system. The telemetry data includes information indicative of the consumer being exposed to the one or more media files while the one or more media files are being played. The operations include creating an electronic fingerprint of the telemetry data and the unique media identifiers of the one or more media files, and storing the electronic fingerprint, the telemetry data, and the unique media identifiers of the one or more media files in a ledger.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating a system for counseling automation providing evidence of concordance, according to various embodiments;

FIG. 2 is a schematic block diagram illustrating an apparatus for counseling automation providing evidence of concordance, according to various embodiments;

FIG. 3 is a schematic block diagram illustrating another apparatus for counseling automation providing evidence of concordance, according to various embodiments;

FIG. 4 is a flowchart diagram illustrating one embodiment of a method for counseling automation providing evidence of concordance, according to various embodiments;

FIG. 5 is a flowchart diagram illustrating one embodiment of a method for uploading media files for counseling automation providing evidence of concordance, according to various embodiments;

FIG. 6 is a flowchart diagram illustrating one embodiment of another method for counseling automation providing evidence of concordance, according to various embodiments;

FIG. 7A is a first part of a flowchart diagram illustrating one embodiment of another method in a medical environment for counseling automation providing evidence of concordance, according to various embodiments; and

FIG. 7B is a second part of the flowchart diagram of FIG. 7A.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims, or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and W hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular N manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C.

An apparatus for counseling automation providing evidence of concordance is disclosed. A method and computer program product also perform the functions of the apparatus. The apparatus includes a processor and non-transitory computer readable storage media storing code. The code is executable by the processor to perform operations that include recording telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system. The telemetry data includes information indicative of the consumer being exposed to the one or more media files while the one or more media files are being played. The operations include creating an electronic fingerprint of the telemetry data and the unique media identifiers of the one or more media files and storing the electronic fingerprint, the telemetry data, and the unique media identifiers of the one or more media files in a ledger.

In some embodiments, the operations include creating a unique identifier for the version of each of the one or more media files and storing the unique identifier for the version of each of the one or more media files and the version of each of the one or more media files in the immutable data storage system. Each unique identifier for a version of a media file of the one or more media files is associated with the version of the media file. In other embodiments, creating the unique identifier for a version of a media file of the one or more media files includes using a hashing function to create a media hash. The unique identifier includes the media hash. In other embodiments, the operations include, prior to creating the electronic fingerprint, serializing the telemetry data into a human readable format.

In some embodiments, creating the electronic fingerprint includes using a hashing function on the telemetry data and the unique media identifiers of the one or more media files to create a session hash. The electronic fingerprint includes the session hash.

In other embodiments, the telemetry data includes keystrokes of the consumer playing the one or more media files, a video of the consumer viewing the one or more media files, consumer responses to media file prompts, consumer personal identification information, and/or a signature by the consumer on a disclosure admission document certifying that the consumer has watched and/or listened to the one or more media files.

In some embodiments, the telemetry data excludes consumer personal identification information and the operations include storing, in an account associated with the consumer, a disclosure admission document in a separate data storage system. The disclosure admission document certifies that the consumer has watched and/or listened to the one or more media files. The disclosure admission document includes the electronic fingerprint and the separate data storage system is separate from the immutable data storage system storing the one or more media files, the telemetry data, and the ledger. In a further embodiment, the operations include storing, in the separate data storage system with the disclosure admission document, a video and/or a photograph of the consumer viewing the one or more media files. In other embodiments, the separate data storage system includes electronic medical records.

In some embodiments, the operations include receiving a ledger identifier for the ledger. The ledger identifier is associated with the electronic fingerprint and the telemetry data and the unique media identifiers of the one or more media files stored on the ledger. In the embodiments, the operations include associating the ledger identifier with the electronic fingerprint and storing the electronic fingerprint, the ledger identifier, and the association between the ledger identifier and the electronic fingerprint on the immutable data storage system. In other embodiments, the consumer playing a media file of the one or more media files includes prohibiting fast forwarding the media file past a point where playing the media file has reached at normal speed. In other embodiments, recording the telemetry data of the consumer playing the one or more media files includes storing the telemetry data in a relational database. In other embodiments, a data storage system storing the ledger is separate from the data storage system storing the versions of the one or more media files and/or the telemetry data.

In some embodiments, the operations further include creating, under direction of a practitioner, a customized disclosure plan for the consumer. The customized disclosure plan includes the one or more media files selected by the practitioner for the consumer. In the embodiments, the operations include transmitting to the consumer a unique login identifier and an associated password, where the unique login identifier and password provide access for playing the one or more media files of the customized disclosure plan.

A method for counseling automation providing evidence of concordance includes recording telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system. The telemetry data includes information indicative of the consumer being exposed to the one or more media files while the one or more media files are being played. The method includes creating an electronic fingerprint of the telemetry data and the unique media identifiers of the one or more media files and storing the electronic fingerprint, the telemetry data and the unique media identifiers of the one or more media files in a ledger.

In some embodiments, the method includes creating a unique identifier for the version of each of the one or more media files and storing the unique identifier for the version of each of the one or more media files and the version of each of the one or more media files in the immutable data storage system. Each unique identifier for a version of a media file of the one or more media files is associated with the version of the media file. In other embodiments, the method includes, prior to creating the electronic fingerprint, serializing the telemetry data into a human readable format. In other embodiments, creating the electronic fingerprint includes using a hashing function on the telemetry data and the unique media identifiers of the one or more media files to create a session hash. The electronic fingerprint includes the session hash.

In some embodiments, the telemetry data includes keystrokes of the consumer playing the one or more media files, a video of the consumer viewing the one or more media files, consumer responses to media file prompts, consumer personal identification information, and/or a signature by the consumer on a disclosure admission document certifying that the consumer has watched and/or listened to the one or more media files. In other embodiments, the telemetry data excludes consumer personal identification information, and the method includes storing, in an account associated with the consumer, a disclosure admission document in a separate data storage system. The disclosure admission document certifies that the consumer has watched and/or listened to the one or more media files. The disclosure admission document includes the electronic fingerprint and the separate data storage system is separate from the immutable data storage system storing the one or more media files, the telemetry data, and the ledger. In other embodiments, the method includes storing, in the separate data storage system with the disclosure admission document, a video and/or a photograph of the consumer viewing the one or more media files, and/or the separate data storage system includes electronic medical records.

In some embodiments, the method includes receiving a ledger identifier for the ledger. The ledger identifier is associated with the electronic fingerprint and the telemetry data and the unique media identifiers of the one or more media files are stored on the ledger. In the embodiments, the method includes associating the ledger identifier with the electronic fingerprint, and storing the electronic fingerprint, the ledger identifier, and the association between the ledger identifier and the electronic fingerprint on the immutable data storage system. In other embodiments, the consumer playing a media file of the one or more media files includes prohibiting fast forwarding the media file past a point where playing the media file has reached at normal speed.

In some embodiments, the method includes creating, under direction of a practitioner, a customized disclosure plan for the consumer. The customized disclosure plan includes the one or more media files selected by the practitioner for the consumer. In the embodiments, the method includes transmitting to the consumer a unique login identifier and an associated password, where the unique login identifier and password provide access for playing the one or more media files.

A program product for counseling automation providing evidence of concordance includes a non-transitory computer readable storage medium storing code. The code is configured to be executable by a processor to perform operations that include recording telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system. The telemetry data includes information indicative of the consumer being exposed to the one or more media files while the one or more media files are being played. The operations include creating an electronic fingerprint of the telemetry data and the unique media identifiers of the one or more media files, and storing the electronic fingerprint, the telemetry data, and the unique media identifiers of the one or more media files in a ledger.

In some embodiments, the operations include creating a unique identifier for the version of each of the one or more media files and storing the unique identifier for the version of each of the one or more media files and the version of each of the one or more media files in the immutable data storage system. Each unique identifier for a version of a media file of the one or more media files is associated with the version of the media file. In other embodiments, the operations include, prior to creating the electronic fingerprint, serializing the telemetry data into a human readable format.

In some embodiments, the telemetry data excludes consumer personal identification information and operations include storing, in an account associated with the consumer, a disclosure admission document in a separate data storage system. The disclosure admission document certifies that the consumer has watched and/or listened to the one or more media files. The disclosure admission document includes the electronic fingerprint and the separate data storage system is separate from the data storage system storing the one or more media files, the telemetry data, and the ledger.

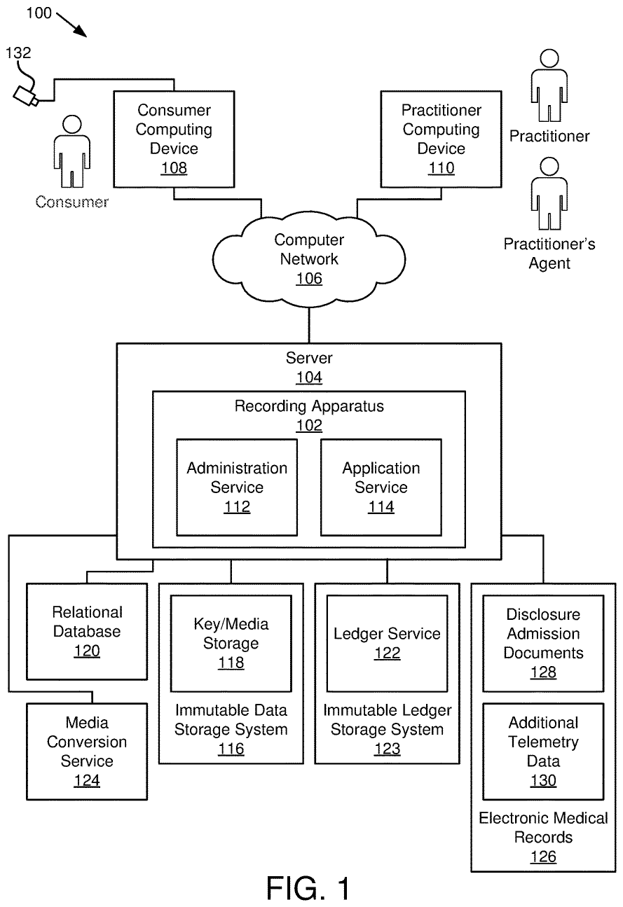

FIG. 1 is a schematic block diagram illustrating a system 100 for counseling automation providing evidence of concordance, according to various embodiments. The system 100 includes a recording apparatus 102 in a server 104 in communication over a computer network 106 with a consumer computing device 108 and a practitioner computing device 110. In some embodiments, the recording apparatus 102 includes an administration service 112 and an application service 114 and the server 104 has access to or is in communication with an immutable data storage system 116 with key and media storage 118, a relational database 120, a ledger service 122 storing ledger information in an immutable ledger storage system 123, and a media conversion service 124. In some embodiments, the system 100 has access to electronic medical records (“EMR”) 126 for storing disclosure admission documents 128 and possibly additional telemetry data 130. The components 102-130 of the system 100 are described below.

The system 100 includes a recording apparatus 102 configured to provide a method of recording and then storing evidence of a disclosure of information to a consumer, as directed by a practitioner, regarding a particular topic, and storing evidence that the consumer viewed, read, listened to, etc. media files pertaining to the topic. The evidence may then be used later to show that the consumer was exposed to the media files and/or acknowledged being exposed to the information.

In some embodiments, the recording apparatus 102 records and stores documentary evidence that the consumer understood the disclosure information. In some embodiments, the recording apparatus 102 records telemetry data of a consumer playing one or more media files where each version of the media file includes information being disclosed to the consumer. Each version of the media file is associated with a unique media identifier and each version of the media file and an associated unique media identifier are stored on an immutable data storage system 116. The telemetry data includes information that indicates the consumer was exposed to the one or more media files while the one or more media files were being played. The recording apparatus 102 creates an electronic fingerprint of the telemetry data and the unique media identifiers of the one or more media files, and stores the electronic fingerprint, the telemetry data, and the unique media identifiers of the one or more media files in a ledger.

In some embodiments, the recording apparatus 102 provides a disclosure admission document that includes the fingerprint and the consumer signs the disclosure admission document. A practitioner stores the signed document in an electronic medical record 126 of the consumer where the electronic medical record 126 is stored separately from the immutable data storage system 116. For medical applications, typically the immutable data storage system 116 does not include personally identifiable information of the consumer where the EMR of the consumer includes the disclosure admission document with the fingerprint and may include personally identifiable information of the consumer. Beneficially, in cases where the consumer claims ignorance of the disclosure information in the form of one or more media files, the immutable data storage system 116 includes evidence that the consumer indeed was exposed to the disclosure information.

The evidence is designed to provide a clear indication that the consumer was exposed to the media files by way of telemetry data recorded as the consumer listened to, read, watched, etc. the media files and provides an electronic fingerprint of the telemetry data and unique identifiers of the version of each media file to which the consumer was exposed.

As used herein the term practitioner is any person that is providing disclosure information on a particular topic and a consumer is any person that is receiving the disclosure information on the particular topic. The practitioner's agent is a person acting on behalf of or under the authority of the practitioner. In some embodiments, the practitioner is a medical doctor, and the consumer is a patient of the medical doctor, and the medical doctor is disclosing medical information to the patient. For example, the doctor may be disclosing information about a medical procedure, a surgery, about prescribed medication, or any other medical topic where the doctor wants the patient to understand risks, expectations, directions, etc. regarding the medical procedure, surgery, medication, etc. The recording apparatus 102 provides a mechanism to document that the patient received and/or understood the medical disclosure.

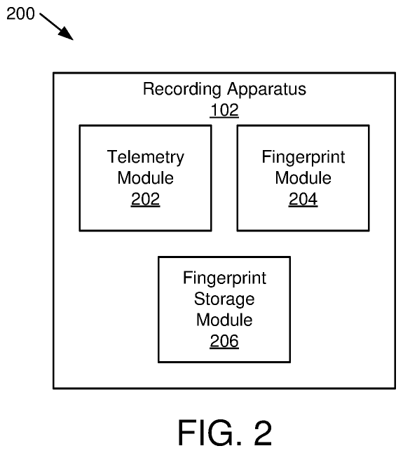

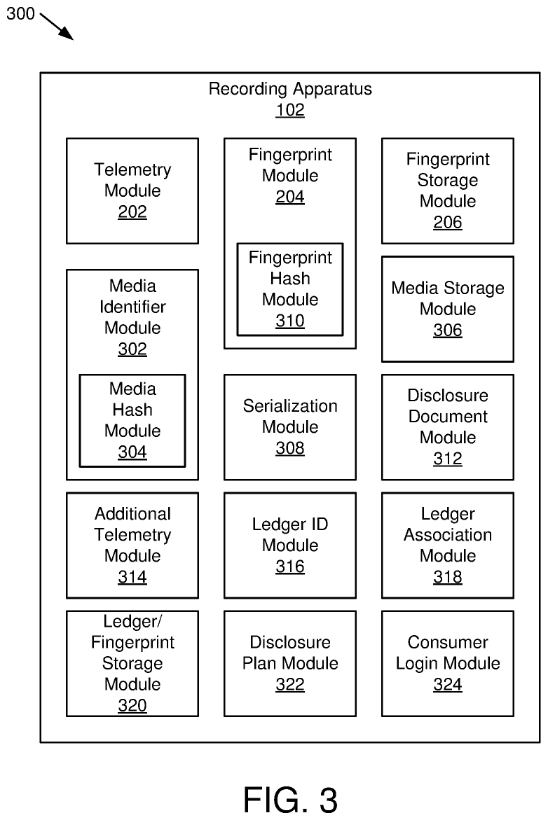

In other embodiments, the practitioner is a government official providing information to the consumer regarding laws, regulations, rules, etc. on a particular topic and the government official wants verification that the consumer received and/or understood the disclosure about the laws, regulations, rules, etc. For example, the practitioner may be a border patrol agent and the consumer may be a person crossing a border where the border patrol agent uses the recording apparatus 102 to document that the border crosser was presented information regarding obligations, rights, responsibilities, etc. pertaining to crossing the border. In other embodiments, the practitioner is a store employee presenting warranty information or other information about a product that a consumer is purchasing. The recording apparatus 102 may be used in any application where a practitioner wants to document that the consumer received a disclosure of information on a particular topic for evidence in a court, a tribunal, a disciplinary proceeding, or other time where the practitioner wants to prove that the consumer received the disclosed information. The recording apparatus 102 is explained in more detail with retards to the apparatuses 200, 300 of FIGS. 2 and 3.

The recording apparatus 102 is depicted in a server 104 in communication with a consumer computing device 108 and a practitioner computing device 110. In some examples, the server 104 hosts a website of the recording apparatus 102 and the computing devices 108, 110 access the website of the recording apparatus 102. In some embodiments, the recording apparatus 102 is an application running on the server 104. In other embodiments, a portion of the recording apparatus 102 runs on consumer computing device 108 and/or the practitioner computing device 110, for example, by way of an application N program interface (“API”). In other embodiments, the consumer is at a location of the practitioner and uses the practitioner computing device 110. In other embodiments, the consumer and/or the practitioner access the server 104 directly. For example, the recording apparatus 102 is running on a server 104 at the location of the practitioner and the consumer is at the location of the practitioner. One of skill in the art will recognize other ways for the consumer and practitioner to access the recording apparatus 102 and other implementations of the recording apparatus 102.

In some embodiments, the server 104 is a server running on a cloud computing service. For example, the server 104 may include a virtual machine (“VM”) running the recording apparatus 102 on a cloud computing service. In other embodiments, the server 104 is a workstation, a desktop computer, a laptop computer, a tablet computer, or the like. The consumer and/or practitioner computing devices 108, 110 are tablet computers, laptop computers, desktop computers, terminals, workstations, a smartphone, or any other computing device capable of interfacing with the recording apparatus 102, capable of playing media files, and recording telemetry data, such as keystrokes, mouse or pointer movement and clicks, verbal responses, video, or any other indicia of the consumer playing, viewing, listening to, reading, etc. the media files.

The computer network 106 is any network or combination of networks that enable communication between the consumer and practitioner computing devices 108, 110 and the server 104. Where the practitioner computing device 110 runs the recording apparatus 102 directly, the computer network 106 provides communication between the consumer computing device 108 and the practitioner computing device 110. The computing device may include a LAN, a WAN, a public network, such as the Internet, a fiber network, a wireless connection or the like and may include a combination of networks.

The wireless connection may be a mobile telephone network. The wireless connection may also employ a Wi-Fi network based on any one of the Institute of Electrical and Electronics Engineers (“IEEE”) 802.11 standards. Alternatively, the wireless connection may be a BLUETOOTH® connection. In addition, the wireless connection may employ a Radio Frequency Identification (“RFID”) communication including RFID standards established by the International Organization for Standardization (“ISO”), the International Electrotechnical Commission (“IEC”), the American Society for Testing and Materials® (“ASTM” ®), the DASH7™ Alliance, and EPCGlobal™.

Alternatively, the wireless connection may employ a ZigBee® connection based on the IEEE 802 standard. In one embodiment, the wireless connection employs a Z-Wave® connection as designed by Sigma Designs®. Alternatively, the wireless connection may employ an ANT® and/or ANT+® connection as defined by Dynastream® Innovations Inc. of Cochrane, Canada.

The wireless connection may be an infrared connection including connections conforming at least to the Infrared Physical Layer Specification (“IrPHY”) as defined by the Infrared Data Association® (“IrDA”®). Alternatively, the wireless connection may be a cellular telephone network communication. All standards and/or connection types include the latest version and revision of the standard and/or connection type as of the filing date of this application.

In some embodiments, the recording apparatus 102 includes an administration service 112 configured to allow the practitioner access to the recording apparatus 102 to change settings, to create a customized disclosure plan for a particular consumer, upload media files, and the like. In various embodiments, the administration service 112 is a mechanism to control various aspects of the recording apparatus 102.

In some embodiments, the recording apparatus 102 includes an application service 114 configured to allow the consumer to play media files of a customized disclosure plan, is configured to record telemetry data of the consumer playing the media files, and to create an electronic signature associated with the telemetry data and unique identifiers of the media files played by the consumer. In various embodiments, the application service 114 accesses the immutable data storage system, the relational database 120, the media conversion service 124, the ledger service 122, etc. The application service 114 is used in various aspects of execution of the recording apparatus 102 to create an immutable record of the consumer being exposed to the disclosure information in the media files of the customized disclosure plan for the consumer. Functions of the application service 114 are disclosed in more detail with regards to the apparatuses 200, 300 of FIGS. 2 and 3.

In some embodiments, the system 100 includes an immutable data storage system 116 that stores media files, a unique identifier for each version of a media file, telemetry data, an electronic fingerprint, and the like. The immutable data storage system 116 is a write once system where data is written and persistently stored in a way that the data is not overwritten. The immutable data storage system 116 is a data storage system where data written to the immutable data storage system 116 is permanently written and can be retrieved as needed in the future. In some embodiments, the immutable data storage system 116 is stored in a way to prevent failure from affecting availability of the data. In some examples, the immutable data storage system 116 uses a random array of independent disks (“RAID”), a backup system, or other means to ensure that failures of drive components do not affect availability of the information.

In some embodiments, the immutable data storage system 116 is located in the server 104. In other embodiments, the immutable data storage system 116 is external to the server 104, such as in a rack where the server 104 occupies a different space in the rack. In other embodiments, the immutable data storage system 116 is available to the server 104 over a network, such as the computer network 106 or other network, such as a storage area network (“SAN”). In some embodiments, the immutable data storage system 116 is available as a subscription service. One of skill in the art will recognize other forms and locations of the immutable data storage system 116.

In some embodiments, the immutable data storage system 116 includes a key and media storage 118 where a practitioner uploads media files and the recording apparatus 102 creates a unique identifier (e.g., key) for each version of the media files. The recording apparatus 102 stores each version of a media file and the associated unique identifier in a way that the unique identifier and version of the media file are associated with each other. In some embodiments, once a version of a media file and associated unique identifier are stored in the immutable data storage system 116, they are not overwritten and are continually accessible.

The system 100 includes a relational database 120 configured to store customized disclosure plans, telemetry data, and other information that will be eventually stored in the immutable data storage system 116. The relational database 120, in some embodiments, is stored in non-volatile data storage of the server 104. The non-volatile data storage may be in the form of solid-state storage, one or more hard disk drives, etc. In some embodiments, the non-volatile data storage is located in the server 104. In other embodiments, the non-volatile data storage is accessible from the server 104. In some examples, the non-volatile data storage is rack-mounted storage connected to a rack-mounted server. In other embodiments, the non-volatile data storage is available to the server 104 over a network, such as a SAN. One of skill in the art will recognize other embodiments of non-volatile data storage of the server 104.

The relational database 120, in some embodiments, stores and provides access to data points that are related to one another. In various embodiments, the relational database 120 includes one or more tables, lists, or other data structures to facilitate storage of telemetry data points, unique identifiers of media files, the media files, etc. in a way that the data is connected. In some embodiments, the relational database 120 is a temporary storage location for data associated with a disclosure session until the data is organized, serialized, etc. and then stored in the immutable data storage system 116.

The system 100 includes a ledger service 122 in an immutable ledger storage system 123 configured to store information from a disclosure session of a consumer in a ledger. As used herein, a ledger is a system for storing transactions in an immutable ledger storage system 123 that is independent of the immutable data storage system 116. Storing disclosure session information in a ledger provides a second, independent storage service to provide additional protection for the disclosure session information. In some embodiments, the immutable ledger storage system 123 is a distributed storage system where copies of the ledger are stored on multiple computing devices. In some embodiments, the multiple computing devices are not under central control but are in a peer group. In other embodiments, the multiple computing devices are unrelated but participate in the ledger service 122.

In some embodiments, the ledger service 122 is a blockchain system and the ledger is a block of the blockchain. A blockchain is a form of a ledger service 122 that is a growing list of records called blocks that are linked together using cryptography. Each block includes a cryptographic hash of the previous block, a timestamp, and transaction data. Typically, a blockchain is extremely difficult to manipulate by an unauthorized person and provides a robust method of maintaining data integrity. Where the ledger service 122 is a blockchain, the ledger service 122 provides a high degree of immutability of the disclosure session data, such as telemetry data, unique identifiers of versions of media files, an electronic signature, etc. related to presentation of disclosure information to the consumer via media files.

In some embodiments, the system 100 includes access to a media conversion service 124 configured to convert media files from one form to another. In some examples, the media conversion service 124 converts media files from one language to another. In other examples, the media conversion service 124 converts media files from one level of resolution to another. In some instances, the media conversion service 124 converts media files presented to a consumer from high resolution to a lower level of resolution to provide reduced storage requirements while providing enough resolution to be used as evidence. In other embodiments, the media conversion service 124 converts media files from one format to another. In some embodiments, the media conversion service 124 runs on the server 104. In other embodiments, the media conversion service 124 is accessible to the server 104 and may be available over a network, such as the computer network 106. One of skill in the art will recognize other ways for the media conversion service 124 to convert media files for the purposes of the recording apparatus 102.

Where the practitioner is a medical provider, in the United States the Health Insurance Portability and Accountability Act (“HIPAA”) is applicable and to avoid applicability of HIPAA audits to the immutable data storage system 116, information in the immutable data storage system 116 is devoid of personal identification information of a patient to which media files of a customized disclosure plan are played. Instead, disclosure session information includes an electronic fingerprint associated specifically with the telemetry data, unique identifiers of the media files played by the patient, etc. of the disclosure session. The practitioner (e.g., doctor) then ensures that the patient signs a disclosure admission document that includes the relevant electronic fingerprint and a statement. The statement typically includes an admission by the patient that the patient has seen, listened to, read, etc. the media files played by the patient and may include that the patient understands the information in the media files. The practitioner saves the disclosure admission document 128 in the electronic medical record of the patient. In some embodiments, the disclosure admission document 128 includes personal identification information of the patient.

In some embodiments, an additional step is added to link the patient to actual viewing, playing, etc. the media files, which is termed here additional telemetry data 130. In some embodiments, the additional telemetry data 130 is stored in the electronic medical record of the patient and includes personal identification of the patient, such as a video recording showing the face of the patient as the patient plays the media files.

In some embodiments, additional equipment is used to verify that the consumer is playing the media files, such as a video camera 132. In medical embodiments, a video camera 132 is often not used as medical patients are sometimes uncomfortable with being recorded in a video. However, in other applications, including some medical applications, a video camera 132 is used by the recording apparatus 102 to gather telemetry data. In some embodiments, telemetry data from a video camera 132 is stored with other telemetry data in the relational database 120 and/or immutable data storage system 116. In other embodiments, other recording equipment (not shown) is used by the recording apparatus 102 to record telemetry data of the consumer, such as a microphone, a signature pad, etc. The telemetry data may take many forms, such as recording keystrokes on a keyboard, consumer responses to media file prompts, consumer personal identification information, a signature by the consumer on a disclosure admission document certifying that the consumer has watched and/or listened to the one or more media files, and the like.

FIG. 2 is a schematic block diagram illustrating an apparatus 200 for counseling automation providing evidence of concordance, according to various embodiments. The apparatus 200 includes a recording apparatus 102 with a telemetry module 202, a fingerprint module 204, and a fingerprint storage module 206, which are described below. In various embodiments, all or a portion of the apparatus 200 is implemented with executable code stored on non-transitory computer readable storage media. The code is executable by a processor. In other embodiments, all or a portion of the apparatus 200 is implemented with a programmable hardware device, such a FPGA, programmable array logic, programmable logic devices, etc. In some embodiments, at least a portion of the apparatus 200 is implemented with hardware circuits.

The apparatus 200 includes a telemetry module 202 configured to record telemetry data of a consumer playing one or more media files. Each media file includes a version of the media file regarding information being disclosed to the consumer and each version of the media file is associated with a unique media identifier. Each version of the media file and an associated unique media identifier are stored on an immutable data storage system 116. The telemetry data includes information indicative of the consumer being exposed to the one or more media files while the one or more media files are being played.

The one or more media files may be in different formats. In one example, a media file is a video and the consumer playing the media file includes playing the video. Where the media file is a video, the consumer being exposed to the media file includes the consumer watching the video and/or listening to sound of the video. In other embodiments, the media file is a sound recording and the consumer playing the media file include the consumer playing the sound recording. Where the media file is a sound recording, the consumer being exposed to the media file includes the consumer listening to the sound recording. In some embodiments, a media file is a document, such as a slide show, a document with text and figures, a hard copy of the document, a booklet, a pamphlet, etc. and the consumer playing the media file and being exposed to the media file includes the consumer viewing the document on a computing device (e.g., 108, 110), reading slides while scrolling through the slides, reading a paper booklet, pamphlet, etc.

In some embodiments, the one or more media files include media files of different types. For example, the one or more media files may include a sound recording integrated with a slide presentation, a sound recording that corresponds to a document, booklet, pamphlet, etc. and the consumer is exposed to the media files by reading or viewing the document, booklet, pamphlet, etc. while listening to the sound recording. One of skill in the art will recognize other forms of the one or more media files and other ways for the consumer to be exposed to the media files.

The telemetry module 202 records telemetry data of the consumer playing one or more media files in various ways. In some embodiments, the telemetry module 202 records keystrokes of the consumer. In some examples, the telemetry module 202 records keystrokes of the consumer accessing a media file of a customized disclosure plan assigned to the consumer, which may include the keystrokes of the consumer clicking on a command to play the media file, pausing play, resuming play, moving backwards in the media file (e.g., rewinding), or other commands related to play of the media file.

In some embodiments, fast forwarding is limited to a point in playback of a media file where play had reached at normal speeds prior to any rewind. For example, if playback of a media file reached the 5 minute mark and the consumer pressed rewind back to the 4 minute mark, fast forward is then limited to between the 4 minute mark and the 5 minute mark but fast forward would not be allowed past 5 minutes until the consumer watches the media file past the 5 minute mark. This ensures that the consumer is exposed to all of the media file running at normal speed at least once and does not allow fast forwarding over portions of the media file that the consumer has not yet watched at normal speed. In some embodiments, the telemetry module 202 controls playback of the media files. In other embodiments, the recording apparatus 102 includes another function that manages playback of the media files.

In other embodiments, the telemetry module 202 records consumer interactions with the media file. For example, the media file may prompt the consumer to provide feedback, answer a question, select an option, or the like and the telemetry module 202 records the consumer's response to the prompt displayed by the media file. In other embodiments, the media file is an online game and the telemetry module 202 records consumer input while playing the game. In the embodiments, one or more of the media files are designed to elicit responses from the consumer at various points while the media files are being played to produce opportunities for the telemetry module 202 to record telemetry data of the consumer. In some embodiments, the telemetry module 202 coordinates recording of telemetry data with playing of the media files on a timeline so that the telemetry data includes timing information that corresponds to specific points during play of the media files.

In other embodiments, the telemetry module 202 records, via a microphone, verbal responses of the consumer. In other embodiments, the telemetry module 202 records video of the consumer while the user is playing the media files, for example, using the video camera 132. In other embodiments, the telemetry module 202 records consumer personal identification information, such as a fingerprint, a retina scan, a signature of the consumer, a photograph of the consumer, personal identification information typed by the consumer, a voiceprint of the consumer, and the like. In some embodiments, the telemetry module 202 records a signature of the consumer on a disclosure admission document certifying that the consumer has watched, listened to, read, etc. the one or more media files.

In some embodiments, the telemetry module 202 associates recorded telemetry data with specific versions of the one or more media files. The media files may include multiple versions and each version includes a unique identifier and the telemetry module 202 associates telemetry data of the consumer playing a particular version of a media file with that version of the media file. In some embodiments, the telemetry module 202 uses the unique identifier of the version of the media file for association with relevant telemetry data. In some embodiments, the telemetry module 202 stores recorded telemetry data in the relational database 120 where the unique identifier of a version of a media file being played is linked to the relevant telemetry data. The relational database 120 provides a convenient mechanism for storage of the telemetry data and associated unique identifiers. In other embodiments, the telemetry module 202 stores the telemetry data in another data structure.

The apparatus 200 includes a fingerprint module 204 configured to create an electronic fingerprint of the telemetry data and the unique media identifiers of the one or more media files. The electronic fingerprint is unique to the telemetry data and unique identifiers of the versions of the media files of a customized disclosure plan for the consumer played by the consumer. The fingerprint module 204 creates a new electronic fingerprint for each new disclosure session with the same or a different consumer. As used herein, a disclosure session refers to a time when the consumer plays media files of a customized disclosure plan. The electronic fingerprint is associated with the relevant telemetry data and unique identifiers of versions of the media files played in the disclosure session where the telemetry data was created.

In some embodiments, the electronic signature is a 32-bit string. In other embodiments, the electronic signature is a 64-bit string. In other embodiments, the electronic signature includes alpha-numeric characters. In other embodiments, the electronic signature includes alpha-numeric characters and other special characters. In some embodiments, the electronic signature is created electronically, but is legible and the practitioner or another person is able to write the electronic signature. In other embodiments, the electronic signature is an electronic file.

In some embodiments, the fingerprint module 204 uses a fingerprinting algorithm to create the electronic fingerprint. In some embodiments, the fingerprinting algorithm is a procedure that maps an arbitrarily large data item (e.g., the telemetry data and unique identifiers) to a shorter bit string, e.g., the electronic fingerprint, that uniquely identifies the original data for all practical purposes.

In some embodiments, the fingerprint module 204 creates the electronic signature using a hash function, as explained further with respect to FIG. 3. In other embodiments, the fingerprint module 204 uses a random number/character generator or other mechanism to create the electronic signature. In other embodiments, the fingerprint module 204 maintains a record of past electronic fingerprints and determines that a new electronic fingerprint does not match past electronic fingerprints. In some embodiments, the fingerprint module 204 creates a unique electronic fingerprint within an organization, a company, etc. that differs from other electronic fingerprints created for the organization, company, etc. One of skill in the art will recognize other ways for the fingerprint module 204 to create electronic fingerprints.

The apparatus 200 includes a fingerprint storage module 206 configured to store the electronic fingerprint, the telemetry data and the unique media identifiers of the one or more media files in a ledger. In some embodiments, the electronic fingerprint is used to locate the telemetry data and unique media identifiers in the ledger. As discussed above, the ledger service 122 stores the ledger on an immutable ledger storage system 123. In some embodiments, the fingerprint storage module 206 stores the electronic fingerprint, the telemetry data, and the unique media identifiers of the one or more media files in a ledger and the ledger service 122 then manages the ledger. In other embodiments, the fingerprint storage module 206 inputs the electronic fingerprint, the telemetry data, and the unique media identifiers to the ledger service 122 and the ledger service 122 stores the electronic fingerprint, the telemetry data, and the unique media identifiers in a ledger. In other embodiments, the fingerprint storage module 206 includes the ledger service 122.

In some embodiments, the ledger is a block of a blockchain. Once the electronic fingerprint, the telemetry data, and the unique media identifiers of the one or more media files are stored in the ledger/blockchain, the ledger service 122, in some embodiments, distributes the ledger/block among multiple data storage devices/computing devices and uses typical blockchain protocol to protect the ledger/blocks from being altered. Beneficially, the apparatus 200 provides a way to record telemetry data of a consumer being exposed to media files of a customized disclosure plan for a consumer, to associate the telemetry data with unique identifiers of the media files, to create a unique electronic fingerprint, and to store the electronic fingerprint, telemetry data, and unique identifiers of the media files in an immutable ledger storage system 123. The apparatus 200 provides evidence that the consumer was exposed to the disclosure information.

FIG. 3 is a schematic block diagram illustrating another apparatus 300 for counseling automation providing evidence of concordance, according to various embodiments. The apparatus 300 includes a telemetry module 202, a fingerprint module 204, and a fingerprint storage module 206 which are substantially similar to those described above in relation to the apparatus 200 of FIG. 2. In various embodiments, the apparatus 300 includes a media identifier module 302 with a media hash module 304, a media storage module 306, a serialization module 308, a fingerprint hash module 310 in the fingerprint module 204, a disclosure document module 312, an additional telemetry module 314, a ledger ID module 316, a ledger association module 318, a ledger/fingerprint storage module 320, a disclosure plan module 322, and/or a consumer login module 324, which are described below. The apparatus 300, in various embodiments, is implemented in a similar way as the apparatus 200 of FIG. 2.

The apparatus 300, in some embodiments, includes a media identifier module 302 configured to create a unique identifier for the version of each of the one or more media files. The media files may each have more than one version so the media identifier module 302 creates a unique identifier for each version of a media file rather than just a single identifier for each media file. The unique identifier, in some embodiments, is structured similarly to the electronic fingerprint and may be a 32-bit string, a 64-bit string, may include alpha-numeric characters, special characters, etc. The unique signature, in some embodiments, is unique to an organization, company, group, etc. In other embodiments, the unique signature is unique across various platforms and may be intended to be unique globally.

In some embodiments, the media identifier module 302 includes a media hash module 304 configured to use a hashing function to create a media hash and the unique identifier includes the media hash. A hash function is a function that may be used to map data of an arbitrary size to fixed-size values. Values returned by a hash function may be called hash values, hash codes, digests, or simply hashes. As used herein, a value returned by a hash function used by the media identifier module 302 is a hash where specifically the value returned by the hash function used by the media hash module 304 is a media hash.

In some embodiments, the media hash module 304 processes a media file using a one-way hash algorithm. In some embodiments, the media hash module 304 uses the Secure Hash Algorithm 2 (“SHA-2”) hash algorithm designed by the United States National Security Agency (“NSA”). In other embodiments, the media hash module 304 uses a similar but different hash algorithm, such as BLAKE3, GOST 34.311-95, HAVAL, Kangaroo Twelve, MD6, or the like. The media hash module 304, in some embodiments, processes a media file bit-by-bit to create a digital fingerprint that is referred to herein as a media hash. The media hash of a version of a media file is unique to that version of the media file and any changes to the media file results in a different media hash.

The apparatus 300, in some embodiments, includes a media storage module 306 configured to store the unique identifier for the version of each of the one or more media files and the version of each of the one or more media files in the immutable data storage system 116. Each unique identifier for a version of a media file of the one or more media files is associated with the version of the media file. Storing each version of the one or more media files and the associated unique identifier in the immutable data storage system 116 provides a way to access the exact version of the media file that was presented to the consumer.

In some embodiments, media storage module 306 stores a full resolution version of each version of a media file. In other embodiments, the media storage module 306 stores one or more lower resolution versions of the media file, which may be used to reduce data storage requirements while still being useful as evidence that the consumer was exposed to the media file.

The apparatus 300, in some embodiments, includes a serialization module 308 configured to, prior to creating the electronic fingerprint, serialize the telemetry data into a human readable format. In some embodiments, the serialization module 308 converts keystrokes, media player commands, responses by the consumer, etc. into a human readable format so that the telemetry data is useful in a court, a hearing, a tribunal, etc. and to judges, administrators, jurors, etc. In some embodiments, the serialization module 308 serializes the telemetry data by providing a timeline of events. In some embodiments, the telemetry data includes photographs, videos, etc. and the serialization module 308 provides labels, information, a title, etc. to the photographs, videos, etc. and may provide a link for viewing the photographs, videos, etc. or for listening to a sound recording. One of skill in the art will recognize other ways for the serialization module 308 module to serialize the telemetry data.

The apparatus 300 includes, in some embodiments, a fingerprint hash module 310 in the fingerprint module 204. The fingerprint hash module 310 is configured to use a hash function on the telemetry data and the unique media identifiers of the one or more media files to create a hash, which in this instance is called a session hash. The electronic fingerprint includes the session hash. In some embodiments, the fingerprint hash module 310 uses the same or similar hash function as the media hash module 304 where the fingerprint hash module 310 processes bit-by-bit the telemetry data and unique signatures of the versions of the media files reviewed by the consumer.

In some embodiments, such as medical applications, the telemetry data excludes consumer personal identification information and the apparatus 300, in some embodiments, includes a disclosure document module 312 configured to store, in an account associated with the consumer, a disclosure admission document in a separate data storage system. In some examples, the disclosure document module 312 stores the disclosure admission document in an electronic medical record of the consumer, which is separate from the relational database 120 or the immutable data storage system 116.

The disclosure admission document certifies that the consumer has watched and/or listened to the one or more media files. In some situations, the disclosure document module 312 presents the disclosure admission document to the consumer for electronic signature. In other embodiments, the practitioner or the practitioner's assistant prints out the disclosure admission document for signature by the consumer and the disclosure document module 312 receives a scan or fax of the signed disclosure admission document.

The disclosure admission document includes the electronic fingerprint, which provides a tie to the electronic signature, telemetry data and unique identifiers of the media files played by or presented to the consumer. In some embodiments, the separate data storage system is separate from the data storage system(s), (e.g., immutable data storage system 116, relational database 120, and/or immutable ledger storage system 123) storing the one or more media files, the telemetry data, and the ledger.

In some examples, such as medical applications, the immutable data storage system 116, the relational database 120, and/or the immutable ledger storage system 123 do not include personally identifiable information about the consumer to avoid HIPAA audits. Having the disclosure admission document of the consumer that includes the electronic fingerprint ties the information stored in an associated ledger and/or immutable data storage system 116 to the consumer and storing the disclosure admission document in an electronic medical record of the consumer ensures that later access of the disclosure admission document provides a pathway to retrieve the telemetry data and unique identifiers of the versions of the media files reviewed by the consumer.

In some embodiments, the apparatus 300 includes an additional telemetry module 314 configured to store, in the separate data storage system with the disclosure admission document, a video and/or a photograph of the consumer viewing the one or more media files. The additional telemetry module 314 stores additional telemetry data that includes personal identification information of the consumer outside of the ledger, the immutable data storage system 116, and/or relational database 120 in this separate data storage system where the disclosure admission document is stored, such as the electronic medical record of the consumer. The additional telemetry data may be a video, photographs, or other telemetry data of the consumer reviewing the media files in the customized disclosure plan of the consumer where the face of the consumer is shown, the voice of the consumer is included, a fingerprint of the consumer is included, etc. Having the additional telemetry data stored with the disclosure admission document provides additional proof that the consumer was exposed to the media files of the customized disclosure plan of the consumer.

The apparatus 300, in some embodiments, includes a ledger ID module 316 configured to receive a ledger identifier from the ledger. The ledger identifier is associated with the electronic fingerprint, the telemetry data, and the unique media identifiers of the one or more media files stored on the ledger. Typically, the ledger service 122 returns a ledger identifier after information is stored in the ledger where the ledger identifier is used to access the information stored on the ledger.

In the embodiments, the apparatus 300 includes a ledger association module 318 configured to associate the ledger identifier with the electronic fingerprint and a ledger/fingerprint storage module 320 configured to store the electronic fingerprint, the ledger identifier, and the association between the ledger identifier and the electronic fingerprint on the immutable data storage system 116. With the ledger identifier and the electronic fingerprint stored on the immutable data storage system 116, the practitioner or others are able to access the ledger where the fingerprint, telemetry data, and unique identifiers are stored.

In some embodiments, the recording apparatus 102 includes an access system configured to allow a user to find a specific electronic fingerprint from information about the disclosure session of the consumer, such as time, location, etc. when the disclosure session occurred. In some embodiments, in non-medical uses, personal identification information is stored with or included in the telemetry data and the access system returns the fingerprint, telemetry data, etc. based on a name or other personal identification information of the consumer. One of skill in the art will recognize other ways that a practitioner or other person can access the telemetry data, unique identifiers of media files, and the associated media files for evidentiary purposes.

The apparatus 300, in some embodiments, includes a disclosure plan module 322 configured to create, under direction of a practitioner, a customized disclosure plan for the consumer. The customized disclosure plan includes the one or more media files selected by the practitioner for the consumer. The practitioner has a purpose for a disclosure for the consumer, which is to disclose information on some topic, as described above. The disclosure plan module 322 allows the practitioner or practitioner's assistant to select certain versions of the media files for the customized disclosure plan.

In some embodiments, the practitioner or practitioner's assistant interacts with the consumer in person to set up a disclosure session with the consumer. For example, the practitioner or practitioner's assistant may provide a tablet computer to the consumer, may position the consumer in front of a monitor, mouse, and keyboard of the practitioner computing device 110, etc. to start the disclosure session. The disclosure session, in some embodiments, is started by the consumer where the consumer plays the one or more media files associated with the customized disclosure plan. In other examples, the practitioner or practitioner's assistant starts playing a media file of the customized disclosure plan.

The apparatus 300, in some embodiments, includes a consumer login module 324 configured to transmit to the consumer a unique login identifier and an associated password where the unique login identifier and password provide access for playing the one or more media files of the customized disclosure plan. The consumer login module 324 provides a mechanism for the consumer to remotely access the one or more media files of the customized disclosure plan. In some embodiments, the consumer accesses a website of the practitioner and then enters the unique login identifier and password to access the media files. In other embodiments, the consumer login module 324, the practitioner, and/or the practitioner's assistant emails, direct messages, etc. the consumer with information to access the media files. In various embodiments, the consumer login module 324, the practitioner, or practitioner's assistant communicates the unique login identifier and password to the consumer.

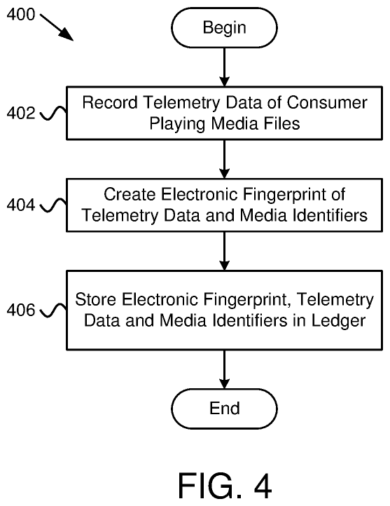

FIG. 4 is a flowchart diagram illustrating one embodiment of a method 400 for counseling automation providing evidence of concordance, according to various embodiments. The method 400 begins and records 402 telemetry data of a consumer playing one or more media files. Each version of a media file of the one or more media files includes information being disclosed to the consumer and is associated with a unique media identifier. Each version of a media file of the one or more media files and an associated unique media identifier are stored on an immutable data storage system 116. The telemetry data includes information indicative of the consumer being exposed to the one or more media files while the one or more media files are being played.

The method 400 creates 404 an electronic fingerprint of the telemetry data and the unique media identifiers of the one or more media files and stores 406 the electronic fingerprint, the telemetry data, and the unique media identifiers of the one or more media files in a ledger, and the method 400 ends. In various embodiments, all or a portion of the method 400 are implemented using the telemetry module 202, the fingerprint module 204, and/or the fingerprint storage module 206.

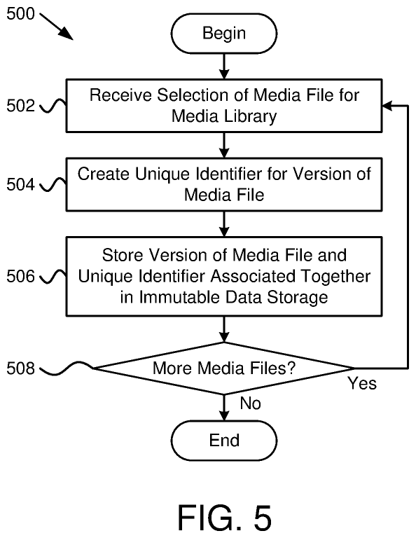

FIG. 5 is a flowchart diagram illustrating one embodiment of a method 500 for uploading media files for counseling automation providing evidence of concordance, according to various embodiments. The method 500 begins and receives 502 a selection of a media file to be uploaded to a media library. The media library, in some embodiments, includes media files on various topics to be disclosed to consumers. In some embodiments, the media files are designed to elicit responses from a consumer to gather telemetry data that ensures that the consumer is paying attention to the media files as they are played.

The method 500 creates 504 a unique identifier for the version the received media file and stores 506 the unique identifier for the version the media file and the version of the media file in the immutable data storage system 116. In some embodiments, the method 500 uses a hashing function to create a media hash and the unique identifier for the version of the media file includes the media hash. The method 500 determines 508 if there are additional media files to upload to the media library. If the method 500 determines 508 that there are additional media files to upload, the method 500 returns and receives a selection of another media file for the media library. If the method 500 determines 508 that there are no additional media files to upload, the method 500 ends. In various embodiments, all or a portion of the method 500 are implemented using the media identifier module 302, the media hash module 304, and/or the media storage module 306.

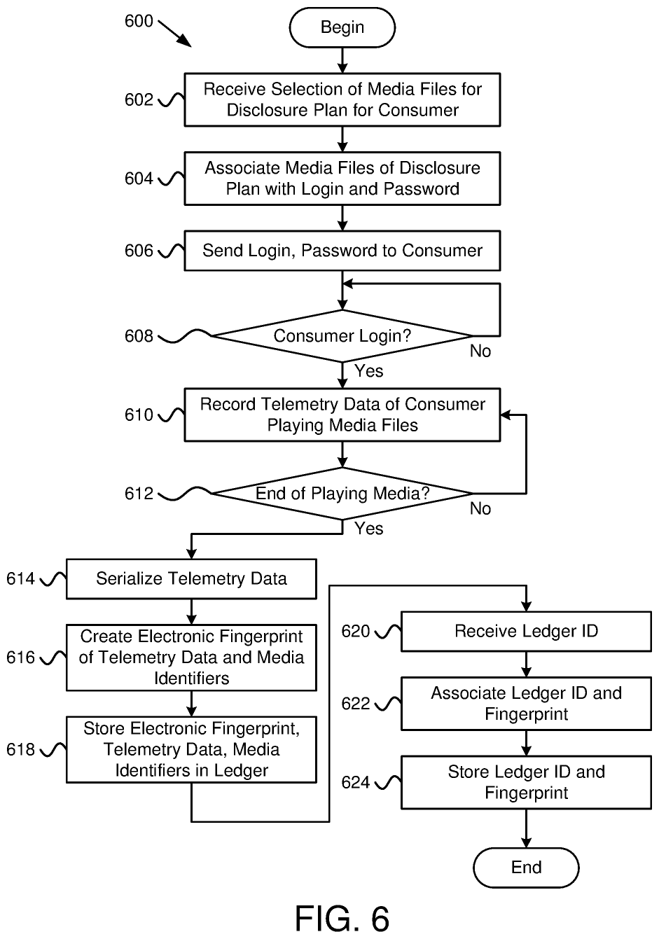

FIG. 6 is a flowchart diagram illustrating one embodiment of another method 600 for counseling automation providing evidence of concordance, according to various embodiments. The method 600 begins and receives 602 selection of a version of one or more media files to be included in a customized disclosure plan for a consumer. In some embodiments, for example where the consumer is remote from the practitioner, the method 600 associates 604 the version of the selected media files with a unique login identifier and password and optionally sends 606 the unique login identifier and password to the consumer. In other embodiments, a practitioner or practitioner's assistant sends or provides the login and password to the consumer. In some embodiments, the method 600 does not associate 604 selected media files with a login or password. For example, where the consumer is at the facility of the practitioner a login and password may not be necessary.

Where the consumer receives the unique login identifier and password, the method 600 determines 608 if the consumer has entered the unique login identifier and password and is logged in. If the method 600 determines 608 that the consumer is not logged in, the method 600 returns and again determines 608 if the consumer has entered the unique login identifier and password and is logged in. If the method 600 determines 608 that the consumer is logged in, the method 600 records 610 telemetry data of the consumer playing the media files of the customized disclosure plan and determines 612 if playing of the media file has ended. If the method 600 determines 612 that playing of the media files has not ended, the method 600 returns and continues to record 610 telemetry data of the consumer playing the media files of the customized disclosure plan. In some embodiments, the telemetry data includes personal identification information of the consumer, such as a signature, a video of the consumer, a voice recording of the consumer, etc. In embodiments where the method 600 does not associate the media files with a login or password, the method 600 skips from receiving 602 a selection of media files to recording 610 telemetry data.

If the method 600 determines 612 that playing of the media files has ended, the method 600 serializes 614 the telemetry data and creates 616 an electronic fingerprint of the telemetry data and the unique media identifiers of the one or more media files of the customized disclosure plan. The method 600 stores 618 the electronic fingerprint, the telemetry data and the unique media identifiers of the one or more media files in a ledger. In some embodiments, the method stores 618 the ledger is in an immutable ledger storage system 123. In other embodiments, the method 600 provides the electronic fingerprint, the telemetry data and the unique media identifiers of the one or more media files to a ledger service 122 for storage in a ledger.

The method 600 receives 620 a ledger identifier for the ledger. The ledger identifier is associated with the electronic fingerprint, the telemetry data and the unique media identifiers of the one or more media files stored on the ledger. The method 600 associates 622 the ledger identifier with the electronic fingerprint and stores 624 the electronic fingerprint, the ledger identifier, and the association between the ledger identifier and the electronic fingerprint on the immutable data storage system 116, and the method 600 ends. In various embodiments, all or a portion of the method 600 are implemented using the telemetry module 202, the fingerprint module 204, the fingerprint storage module 206, the media identifier module 302, the media hash module 304, the media storage module 306, the serialization module 308, the fingerprint hash module 310, the disclosure document module 312, the additional telemetry module 314, the ledger ID module 316, the ledger association module 318, the ledger/fingerprint storage module 320, the disclosure plan module 322, and/or the consumer login module 324.

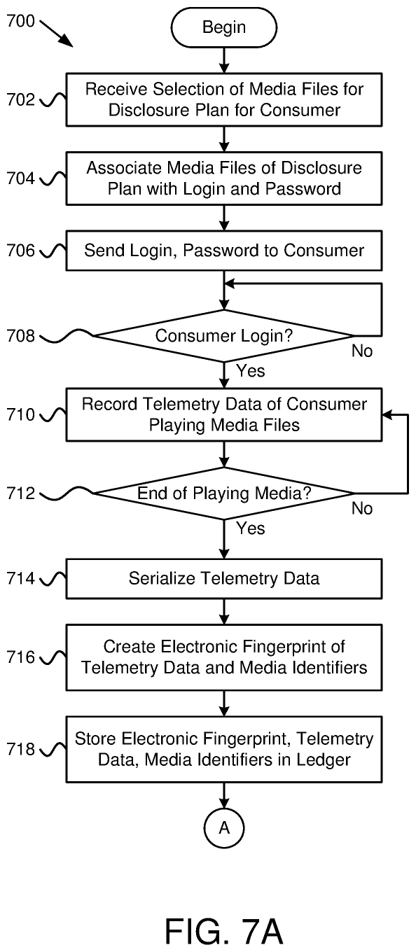

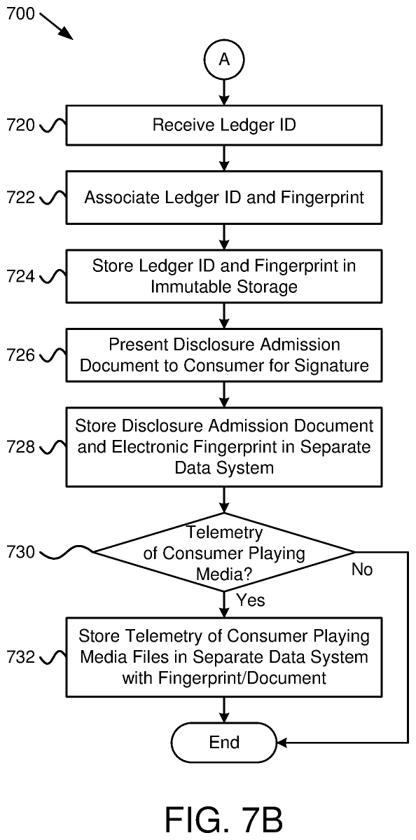

FIG. 7A is a first part and FIG. 7B is a second part of a flowchart diagram illustrating one embodiment of another method 700 in a medical environment for counseling automation providing evidence of concordance, according to various embodiments. The method 700 begins and receives 702 selection of a version of one or more media files to be included in a customized disclosure plan for a consumer. In some embodiments, for example where the consumer is remote from the practitioner, the method 700 associates 704 the version of the selected media files with a unique login identifier and password and optionally sends 706 the unique login identifier and password to the consumer. In other embodiments, a practitioner or practitioner's assistant sends the login and password to the consumer. In some embodiments, the method 700 does not associate 704 selected media files with a login or password. For example, where the consumer is at the facility of the practitioner, such as at a doctor's office, a login and password may not be necessary.

Where the consumer receives the unique login identifier and password, the method 700 determines 708 if the consumer has entered the unique login identifier and password and is logged in. If the method 700 determines 708 that the consumer is not logged in, the method 700 returns and again determines 708 if the consumer has entered the unique login identifier and password and is logged in. If the method 700 determines 708 that the consumer is logged in, the method 700 records 710 telemetry data of the consumer playing the media files of the customized disclosure plan and determines 712 if playing of the medias file has ended. If the method 700 determines 712 that playing of the media files has not ended, the method 700 returns and continues to record 710 telemetry data of the consumer playing the media files of the customized disclosure plan. In some embodiments, the telemetry data includes personal identification information of the consumer, such as a signature, a video of the consumer, a voice recording of the consumer, etc. In embodiments where the method 700 does not associate the media files with a login or password, the method 700 skips from receiving 702 a selection of media files to recording 710 telemetry data.

If the method 700 determines 712 that playing of the media files has ended, the method 700 serializes 714 the telemetry data and creates 716 an electronic fingerprint of the telemetry data and the unique media identifiers of the one or more media files of the customized disclosure plan. The method 700 stores 718 the electronic fingerprint, the telemetry data and the unique media identifiers of the one or more media files in a ledger. In some embodiments, the method stores 718 the ledger is in an immutable ledger storage system 123. In other embodiments, the method 700 provides the electronic fingerprint, the telemetry data and the unique media identifiers of the one or more media files to a ledger service 122 for storage in a ledger.

The method 700 receives 720 (follow “A” on FIG. 7A to “A” on FIG. 7B) a ledger identifier for the ledger. The ledger identifier is associated with the electronic fingerprint, the telemetry data and the unique media identifiers of the one or more media files stored on the ledger. The method 700 associates 722 the ledger identifier with the electronic fingerprint and stores 724 the electronic fingerprint, the ledger identifier, and the association between the ledger identifier and the electronic fingerprint on the immutable data storage system 116. The method 700 presents 726 a disclosure admission document to the consumer for signature.

Once the consumer has signed the disclosure admission document, the method 700 stores 728, in an account associated with the consumer, the signed disclosure admission document in a separate data storage system. The separate data storage system is separate from the immutable data storage system storing the one or more media files, the telemetry data, and the ledger. In some embodiments, the method 700 stores 728 the signed disclosure admission document in an electronic media record of the consumer. The disclosure admission document certifies that the consumer has watched and/or listened to the one or more media files and the disclosure admission document includes the electronic fingerprint.

The method 700 determines 730 if there is additional telemetry data with personal identification information of the consumer playing the media files. If the method 700 determines 730 that there is additional telemetry data with personal identification information of the consumer playing the media files, the method 700 stores 732 the additional telemetry data in the separate data storage system with the fingerprint and disclosure admission document, for example, in an electronic medical record of the consumer. If the method 700 determines 730 that there is no additional telemetry data, the method 700 ends. In some embodiments, the method 700 may be used in a non-medical environment and the disclosure admission document and additional telemetry data is stored in the immutable data storage system 116.

In various embodiments, all or a portion of the method 600 are implemented using the telemetry module 202, the fingerprint module 204, the fingerprint storage module 206, the media identifier module 302, the media hash module 304, the media storage module 306, the serialization module 308, the fingerprint hash module 310, the disclosure document module 312, the additional telemetry module 314, the ledger ID module 316, the ledger association module 318, the ledger/fingerprint storage module 320, the disclosure plan module 322, and/or the consumer login module 324.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
a processor; and
non-transitory computer readable storage media storing code, the code being executable by the processor to perform operations comprising:
recording telemetry data of a consumer playing a version of one or more media files, each version of a media file of the one or more media files comprising information being disclosed to the consumer and is associated with a unique media identifier, each version of a media file of the one or more media files and an associated unique media identifier stored on an immutable data storage system, the telemetry data comprising information indicative of the consumer being exposed to the one or more media files while the one or more media files are being played;
creating an electronic fingerprint of the telemetry data and the unique media identifiers of the one or more media files; and
storing the electronic fingerprint, the telemetry data and the unique media identifiers of the one or more media files in a ledger.

2. The apparatus of claim 1, wherein the operations further comprise:
creating a unique identifier for the version of each of the one or more media files; and
storing the unique identifier for the version of each of the one or more media files and the version of each of the one or more media files in the immutable data storage system,
wherein each unique identifier for a version of a media file of the one or more media files is associated with the version of the media file.

3. The apparatus of claim 2, wherein creating the unique identifier for a version of a media file of the one or more media files comprises using a hashing function to create a media hash, wherein the unique identifier comprises the media hash.

4. The apparatus of claim 1, wherein the operations further comprise, prior to creating the electronic fingerprint, serializing the telemetry data into a human readable format.

5. The apparatus of claim 1, wherein creating the electronic fingerprint comprises using a hashing function on the telemetry data and the unique media identifiers of the one or more media files to create a session hash, the electronic fingerprint comprising the session hash.

6. The apparatus of claim 1, wherein the telemetry data comprises:
keystrokes of the consumer playing the one or more media files;
a video of the consumer viewing the one or more media files;
consumer responses to media file prompts;
consumer personal identification information; and/or
a signature by the consumer on a disclosure admission document certifying that the consumer has watched and/or listened to the one or more media files.

7. The apparatus of claim 1, wherein the telemetry data excludes consumer personal identification information and wherein the operations further comprise storing, in an account associated with the consumer, a disclosure admission document in a separate data storage system, the disclosure admission document certifying that the consumer has watched and/or listened to the one or more media files, the disclosure admission document comprises the electronic fingerprint, the separate data storage system separate from the immutable data storage system storing the one or more media files, the telemetry data, and the ledger.

8. The apparatus of claim 7, wherein the operations further comprise storing, in the separate data storage system with the disclosure admission document, a video and/or a photograph of the consumer viewing the one or more media files.

9. The apparatus of claim 7, wherein the separate data storage system comprises electronic medical records.

10. The apparatus of claim 1, wherein the operations further comprise:
receiving a ledger identifier for the ledger, the ledger identifier associated with the electronic fingerprint, the telemetry data and the unique media identifiers of the one or more media files stored on the ledger;
associating the ledger identifier with the electronic fingerprint; and
storing the electronic fingerprint, the ledger identifier, and the association between the ledger identifier and the electronic fingerprint on the immutable data storage system.

11. The apparatus of claim 1, wherein the consumer playing a media file of the one or more media files comprises prohibiting fast forwarding the media file past a point where playing the media file has reached at normal speed.

12. The apparatus of claim 1, wherein recording the telemetry data of the consumer playing the one or more media files comprises storing the telemetry data in a relational database.

13. The apparatus of claim 1, wherein a data storage system storing the ledger is separate from the data storage system storing the versions of the one or more media files and/or the telemetry data.

14. The apparatus of claim 1, wherein the operations further comprise:
creating, under direction of a practitioner, a customized disclosure plan for the consumer, the customized disclosure plan comprising the one or more media files selected by the practitioner for the consumer; and
transmitting to the consumer a unique login identifier and an associated password, wherein the unique login identifier and password provide access for playing the one or more media files of the customized disclosure plan.

15. A method comprising:
recording telemetry data of a consumer playing one or more media files, each version of a media file of the one or more media files comprising information being disclosed to the consumer and is associated with a unique media identifier, each version of a media file of the one or more media files and an associated unique media identifier stored on an immutable data storage system, the telemetry data comprising information indicative of the consumer being exposed to the one or more media files while the one or more media files are being played;
creating an electronic fingerprint of the telemetry data and the unique media identifiers of the one or more media files; and
storing the electronic fingerprint, the telemetry data and the unique media identifiers of the one or more media files in a ledger.

16. The method of claim 15, further comprising:
creating a unique identifier for the version of each of the one or more media files; and
storing the unique identifier for the version of each of the one or more media files and the version of each of the one or more media files in the immutable data storage system,
wherein each unique identifier for a version of a media file of the one or more media files is associated with the version of the media file.

17. The method of claim 15, further comprising, prior to creating the electronic fingerprint, serializing the telemetry data into a human readable format.

18. The method of claim 15, wherein creating the electronic fingerprint comprises using a hashing function on the telemetry data and the unique media identifiers of the one or more media files to create a session hash, the electronic fingerprint comprising the session hash.

19. The method of claim 15, wherein the telemetry data comprises:
keystrokes of the consumer playing the one or more media files;
a video of the consumer viewing the one or more media files;
consumer responses to media file prompts;
consumer personal identification information; and/or
a signature by the consumer on a disclosure admission document certifying that the consumer has watched and/or listened to the one or more media files.

20. The method of claim 15, wherein the telemetry data excludes consumer personal identification information and further comprising storing, in an account associated with the consumer, a disclosure admission document in a separate data storage system, the disclosure admission document certifying that the consumer has watched and/or listened to the one or more media files, the disclosure admission document comprises the electronic fingerprint, the separate data storage system separate from the immutable data storage system storing the one or more media files, the telemetry data, and the ledger.

21. The method of claim 20, further comprising storing, in the separate data storage system with the disclosure admission document, a video and/or a photograph of the consumer viewing the one or more media files, and/or wherein the separate data storage system comprises electronic medical records.

22. The method of claim 15, further comprising:
receiving a ledger identifier for the ledger, the ledger identifier associated with the electronic fingerprint, the telemetry data and the unique media identifiers of the one or more media files stored on the ledger;
associating the ledger identifier with the electronic fingerprint; and
storing the electronic fingerprint, the ledger identifier, and the association between the ledger identifier and the electronic fingerprint on the immutable data storage system.

23. The method of claim 15, wherein the consumer playing a media file of the one or more media files comprises prohibiting fast forwarding the media file past a point where playing the media file has reached at normal speed.

24. The method of claim 15, further comprising:
creating, under direction of a practitioner, a customized disclosure plan for the consumer, the customized disclosure plan comprising the one or more media files selected by the practitioner for the consumer; and
transmitting to the consumer a unique login identifier and an associated password, wherein the unique login identifier and password provide access for playing the one or more media files.

25. A program product comprising a non-transitory computer readable storage medium storing code, the code being configured to be executable by a processor to perform operations comprising:
recording telemetry data of a consumer playing one or more media files, each version of a media file of the one or more media files comprising information being disclosed to the consumer and is associated with a unique media identifier, each version of a media file of the one or more media files and an associated unique media identifier stored on an immutable data storage system, the telemetry data comprising information indicative of the consumer being exposed to the one or more media files while the one or more media files are being played;
creating an electronic fingerprint of the telemetry data and the unique media identifiers of the one or more media files; and
storing the electronic fingerprint, the telemetry data and the unique media identifiers of the one or more media files in a ledger.

26. The program product of claim 25, wherein the operations further comprise:
creating a unique identifier for the version of each of the one or more media files; and
storing the unique identifier for the version of each of the one or more media files and the version of each of the one or more media files in the immutable data storage system,
wherein each unique identifier for a version of a media file of the one or more media files is associated with the version of the media file.

27. The program product of claim 25, wherein the operations further comprise, prior to creating the electronic fingerprint, serializing the telemetry data into a human readable format.

28. The program product of claim 25, wherein the telemetry data excludes consumer personal identification information and wherein operations further comprise storing, in an account associated with the consumer, a disclosure admission document in a separate data storage system, the disclosure admission document certifying that the consumer has watched and/or listened to the one or more media files, the disclosure admission document comprises the electronic fingerprint, the separate data storage system separate from the data storage system storing the one or more media files, the telemetry data, and the ledger.
